(19) United States
(12) Patent Application Publication
(10) Pub. No.: US 2013/0302825 A1
(43) Pub. Date: Nov. 14, 2013

(54) USES OF MACROPHAGE MANNOSE RECEPTOR TO SCREEN COMPOUNDS AND USES OF THESE COMPOUNDS

(71) Applicants: Sylaja Murikipudi, Medford, MA (US); Thomas M. Lancaster, Stoneham, MA (US); Todd C. Zion, Marblehead, MA (US)

(72) Inventors: Sylaja Murikipudi, Medford, MA (US); Thomas M. Lancaster, Stoneham, MA (US); Todd C. Zion, Marblehead, MA (US)

(73) Assignee: Merck, P.O. BOX 2000, NJ (US)

(21) Appl. No.: 13/880,544

(22) PCT Filed: Sep. 27, 2011
(86) PCT No.: PCT/US11/53390

§ 371 Date: Jul. 30, 2013

Related U.S. Application Data
(60) Provisional application No. 61/393,007, filed on Oct. 14, 2010.

(51) Int. Cl. G01N 33/53

(52) U.S. Cl. CPC G01N 33/5308
USPC 435/7.21

(57) ABSTRACT

Methods and associated compositions of matter (e.g., kits, cell lines, etc.) for screening compounds that bind to macrophase mannose receptor (MMR). Compounds identified by these methods and drug conjugates that includes these compounds are also encompassed as are their uses in the manufacture of medicaments.

### BACKGROUND

Lectins are proteins which recognize and bind specific carbohydrates, or patterns of Carbohydrates.¹ There are many different classes of lectins with different structures and functions. Typical functions of lectins include binding to carbohydrates found on the surface of pathogens such as bacteria or yeast in order to elicit an immune response.²

Common classes of lectins found in animals include C type lectins, which depend on calcium for their binding ability, S type lectins which bind to sulfhydryl or β-galactoside groups, and P type lectins, which bind to phosphomannosyl groups.² These classes can be further subdivided based on their specific structures, binding abilities and functions. For example, C type lectins all contain a specific type of carbohydrate binding domain known as a “C type lectin-like domain”, or CTLD. However, the various subclasses of C type lectins encompass soluble and cell based receptors, molecules with single or multiple CTLDs, and have differing affinities for various sugars.^{2, 3}

One key subclass of C type lectins are Group III C-type lectins, or collectins. Collectins are soluble proteins which have a single C type recognition domain and a collagenous domain. They are capable of forming oligomers with a higher avidity for specific carbohydrate domains than the monomeric form.² Common collectins include Mannose Binding Lectin (MBL), which can directly and indirectly activate the complement system. Surfactant Proteins-A and -D are found mainly in the lungs and bind to a variety of pathogens. Unlike MBL, SP-A and SP-D cannot directly activate the complement system; they can act as opsonins as well as cause aggregation of pathogens, altering their ability to be phagocytosed.⁴

Another key subclass of C type lectins are Group VI C type lectins, known as the mannose receptor family. This group of transmembrane lectins is defined by its multiple CTLDs, N-terminal cysteine rich domain, and fibronectin type II domain. The prototypical member of this family is the macrophage mannose receptor (MMR) although there are several other members of the mannose receptor family, including the PLA2 receptor, DEC-205, and ENDO180.³ Like the collectins, a main function of MMR is to recognize pathogens via their surface glycosylation. The receptors constitutively recycle between the cell surface and the interior of the cell. Bound molecules are transported first to endosomes and then on to lysosomes for degradation (as part of the innate immune response) or are presented on the cell surface via the MHC receptors for activation of the humoral immune response.⁵ MMR recognizes several different patterns of carbohydrate, preferentially binding to terminal mannose, L-fucose, and N-acetylglucosamine residues, binding glucose to a lower degree, and showing little if any affinity for galactose.⁶ MMR was first discovered on macrophages, but it is also found in some amount on other cell types, including dendritic cells, lymphatic and liver sinusoidal endothelial cells, retinal pigment epithelium, kidney mesangial cells, and tracheal smooth muscle cells.³

### BRIEF DESCRIPTION OF THE DRAWING

FIG. 1a is a graph of the uptake of a labeled insulin-saccharide conjugate (Alexa488-SI-0052) in NR8383 rat alveolar macrophages in the absence of mannan and in the presence of 5 mg/mL mannan. Mannose specific uptake is shown as the difference between total uptake (without mannan) and the uptake in the presence of mannan (non-specific uptake).

FIG. 1b is a graph of the uptake of a labeled insulin-saccharide conjugate (Alexa488-SI-0052) in NR8383 rat alveolar macrophages in the presence of different concentrations of α-methyl mannose (α-MM), glucose and galactose.

FIG. 2 is a graph of the uptake of a labeled ovalbumin (FITC-ovalbumin) in NR8383 rat alveolar macrophages in the presence of different concentrations of three different insulin-saccharide conjugates (SI-0052, SI-0047 and SI-0048 whose structures are shown in FIG. 5).

FIG. 3 is a graph of the uptake of a labeled ovalbumin (FITC-ovalbumin) in NR8383 rat alveolar macrophages in the presence of an insulin-saccharide conjugate (SI-0047) and different concentrations of unconjugated insulin (RHI).

FIG. 4 is a graph showing the levels of different cytokines (IL-1β, IL-10, etc.) after NR8383 rat alveolar macrophages were incubated with different concentrations of an insulin-saccharide conjugate (SI-0052).

FIG. 5 shows the structures of exemplary insulin-saccharide conjugates SI-0052, SI-0047 and SI-0048. The symbol “insulin” inside an oval as shown in FIG. 5 is intended to represent wild-type human insulin.

### SUMMARY

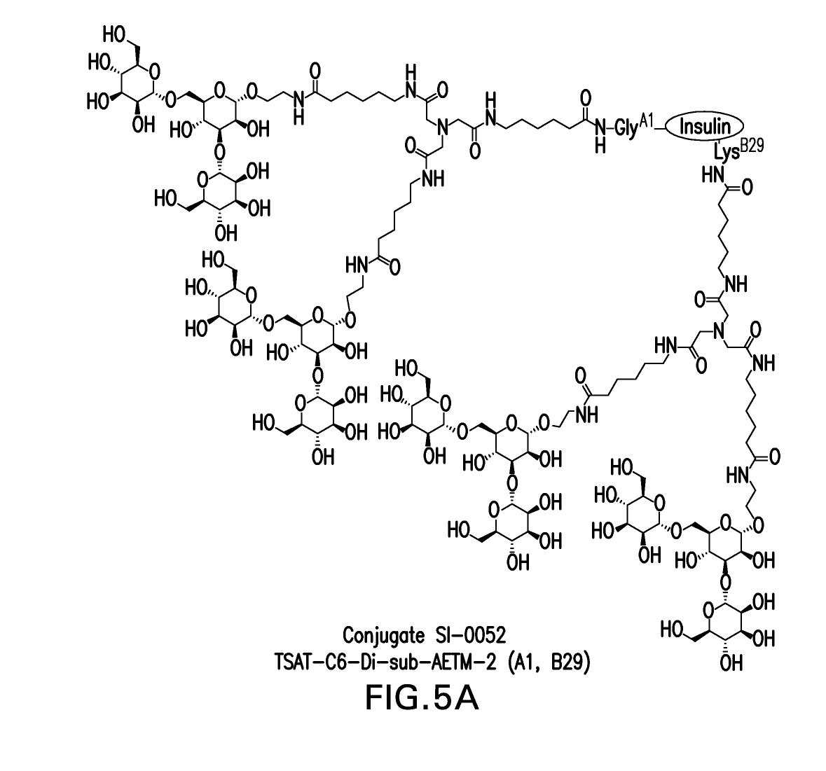

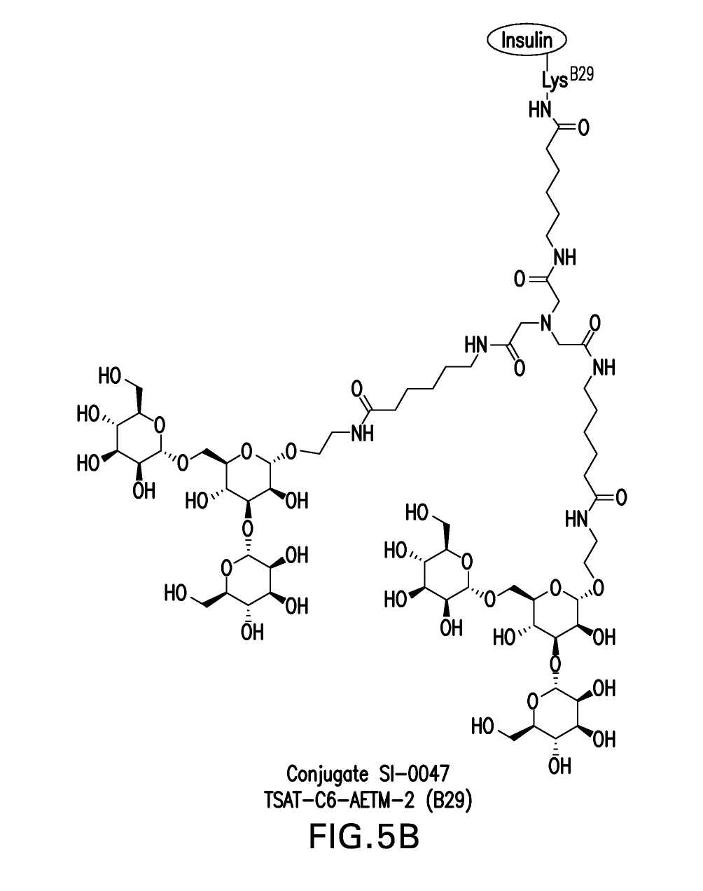

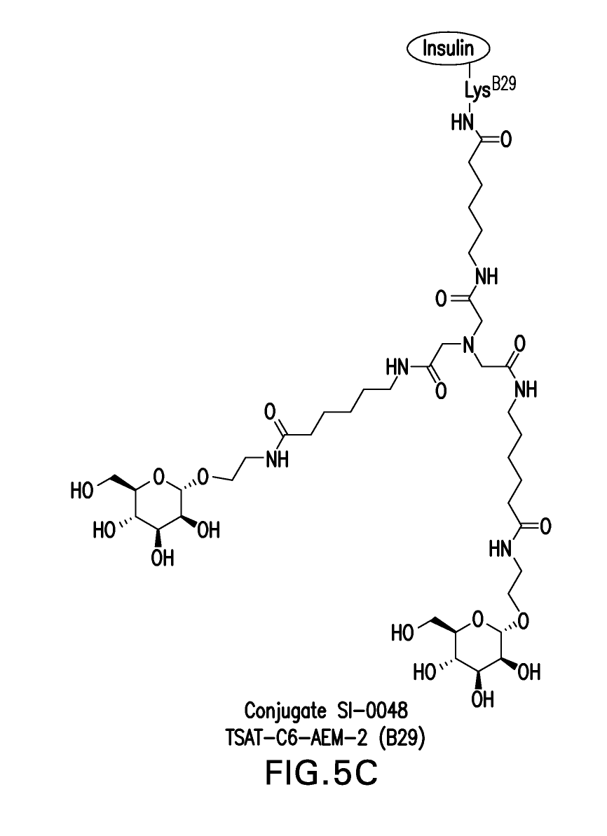

We have recently demonstrated that when certain saccharides are conjugated to a drug (e.g., an insulin molecule) and administered to a subject (e.g., a rat, a mini-pig, etc.) the resulting conjugate exhibits pharmacokinetic (PK) and pharmacodynamic (PD) properties that vary with systemic glucose concentration (e.g., see WO 2010/88294 which is incorporated herein by reference in its entirety). In particular, we have identified classes of drug-saccharide conjugates that exhibit longer lifetimes under hyperglycemic conditions than under hypoglycemic conditions. In light of these properties, these “glucose-responsive” drug-saccharide conjugates have a greater effect on the patient when glucose concentrations are high than when they are low. This is particularly useful when the drug is an insulin molecule since insulin is only needed by the subject under hyperglycemic conditions and would in fact have a negative impact if it exerted an effect under hypoglycemic conditions. Some exemplary insulin-saccharide conjugates are shown in FIG. 5. As discussed in WO 2010/88294, the conjugates are also useful for drugs other than insulin.

We have previously postulated that the “glucose-responsive” nature of these conjugates may result from binding between the saccharide components of the conjugates and one or more endogenous lectins in the subject. The present disclosure describes experiments that have allowed us for the first time to identify one of these endogenous lectins. By identifying a relevant endogenous lectin we can now use this endogenous lectin in assays to screen other compounds (not necessarily saccharides) for their ability to bind with the endogenous lectin. In particular we can now screen different compounds for their binding affinities with this endogenous lectin and also assess how this binding is affected by different concentrations of glucose. Based on our results we can now also select compounds that are known to bind this endogenous lectin (e.g., based on previous studies) and include these in an inventive conjugate. The present disclosure encompasses these other compounds and their use as components of inventive conjugates. We can also use this information to identify other compounds (again not necessarily saccharides) that can inhibit the binding between previously identified conjugates (or their saccharide components) and this endogenous lectin. These other compounds may be useful as modulators of the interactions between a drug-saccharide conjugate and the endogenous lectin. The present disclosure encompasses these other compounds and their uses as modulators of inventive conjugates. The present disclosure also encompasses these screening methods and associated compositions of matter, e.g., kits, cell lines, etc. that can be used to perform the screening methods.

### EXAMPLES

### Example 1a

Alexa488-SI-0052 was prepared by reacting 276 nmol SI-0052 (an exemplary insulin-saccharide conjugate whose structure is shown in FIG. 5) with 1 mg Alexa488 Succinimidyl Ester (Invitrogen) in 667 μl 0.1M sodium bicarbonate buffer, pH=8.3, with constant stirring for 1 hour at room temperature. SI-0052 was prepared as described in WO 2010/88294 (see methods that were used to make conjugate II-2 or TSAT-C6-Di-sub-AETM-2 (A1,B29) in Example 76). Labeled SI-0052 was separated from unreacted dye using 6 kDa NMWCO desalting columns (Pierce). Fractions containing SI-0052 (as determined by absorbance at 280 nM) were pooled and concentrated using 3000 Da NMWCO centrifugal concentrators (Millipore). Concentration of SI-0052 was determined using a BCA total protein assay (Pierce).

NR8383 rat alveolar macrophages were obtained from ATCC and cultured in gelatin coated flasks in F12K medium +15% heat inactivated FBS+antibiotics. For uptake experiments, NR8383 were seeded in gelatin coated 96 well plates and allowed to reach confluence. Cells were washed 1× with PBS and incubated for 1 hour (at 37° C., 5% CO2) with varying concentrations of Alexa488 SI-0052 (in HEPES buffered saline [pH=7.4] containing 1% BSA, 0.1% HI FBS, 2 mM Ca2+, and 0.5 mM Mg2+). Each condition was carried out in triplicate. Each concentration of Alexa488-SI-0052 was tested with and without the presence of 5 mg/mL mannan, which is known to block binding by the mannose receptor.⁷ After incubation, SI-0052 solution was replaced with 5 mM EDTA in cold PBS and cells placed on ice for 10 minutes. Cells were transferred to V-bottom 96 well plates and centrifuged (800 g, 7 min, 4C) to collect. Pellets were washed with cold 5 mM EDTA and again centrifuged. Cells were then resuspended in 1% paraformaldehyde in PBS and stored at 4C in the dark until analysis.

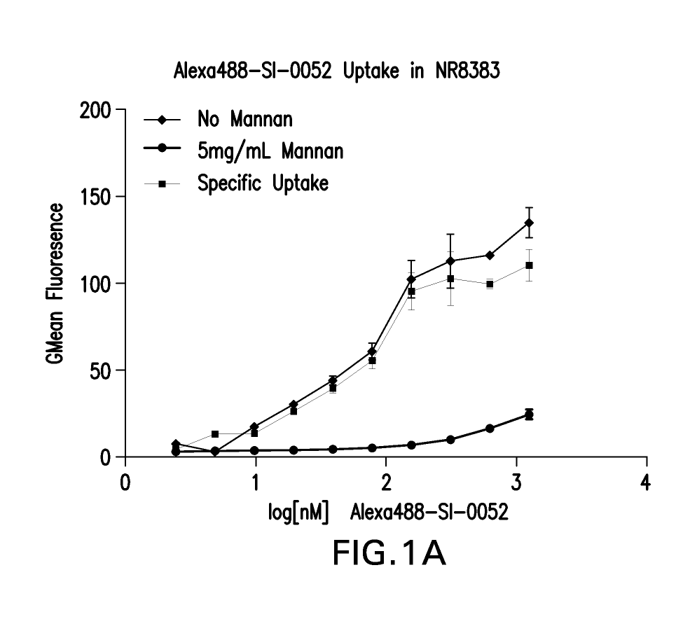

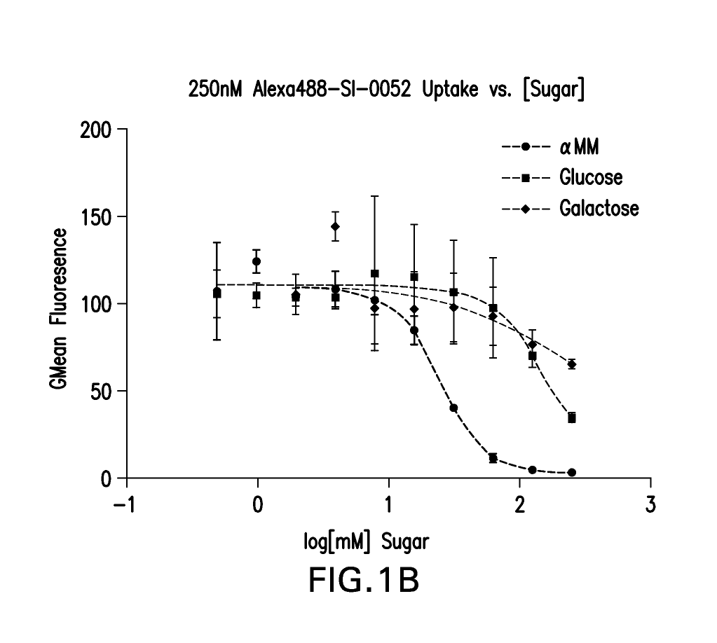

Uptake of Alexa488-SI-0052, as measured by cellular fluorescence, was assessed using flow cytometry (FACSCalibur). The geometric mean of fluorescence for 5000-10000 cells was measured for each sample. Mannose specific uptake was taken to be the difference between total uptake (without mannan) and the uptake in the presence of mannan (non-specific uptake). As shown in FIG. 1a, most Alexa488-SI-0052 incorporation by NR8383 is blocked by the presence of mannan, suggesting that the mannose receptor plays a key role in its uptake. Similar data was collected for other conjugates (Alexa488-SI-0047 and Alexa488-SI-0048, prepared as with Alexa488-SI-0052 but using the SI-0047 and SI-0048 conjugates whose structures are also shown in FIG. 5) as well as FITC-Ovalbumin (a mannosylated protein known to be a ligand for the mannose receptor, purchased from Invitrogen). SI-0047 and SI-0048 were prepared in accordance with methods that are disclosed in WO 2010/88294.

### Example 1b

The uptake of Alexa488-SI-0052 was measured, as described above, in the presence of various sugars known to have varying affinities for the mannose receptor. NR8383 were incubated with a constant concentration of Alexa488-SI-0052 (250 nM, chosen because this concentration lies on the concentration dependent portion of the Alexa488-SI-0052 uptake curve) and varying concentrations of α-methyl mannose (α-MM), glucose and galactose.

Sugars with greater affinity for the receptor involved in Alexa488-SI-0052 uptake will cause a decrease in Alexa488-SI-0052 uptake at lower concentrations than sugars with a lower affinity. The data in FIG. 1b indicate that α-MM interferes the most with Alexa488-SI-0052 uptake, followed by glucose and then galactose. This compares well with the known rank order affinity of MMR for these sugars.⁶

### Example 2

Ovalbumin is a known ligand of MMR.⁷ Therefore, FITC-ovalbumin was used as a marker of uptake by this receptor. NR8383 were incubated, as described above, with a fixed concentration of FITC-ovalbumin (250 nM, on the concentration dependent portion of it uptake curve) in the presence of varying amounts of unlabeled conjugates. It is expected that conjugates with greater affinity for MMR (the pathway by which FITC-ovalbumin is internalized) will inhibit FITC-ovalbumin uptake at lower concentrations than those with a lower affinity for MMR.

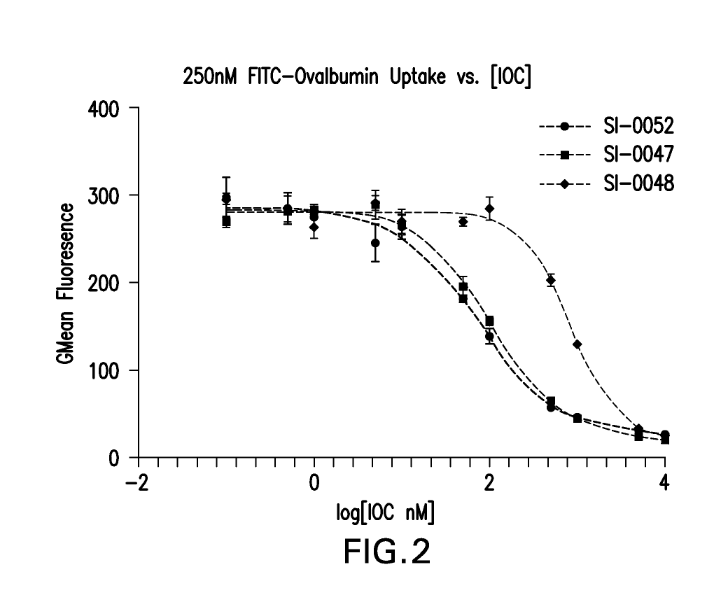

The data in FIG. 2 show that various conjugates inhibit FITC-ovalbumin uptake differently. The IC50 of the various conjugates ranges from 815 nM for SI-0048, to 105 nM for SI-0047 to 76 nM for SI-0052. Comparing the IC50s of various conjugates offers a way to assess their relative affinities for the mannose receptor, without the need for derivitization of the conjugate constructs.

### Example 3

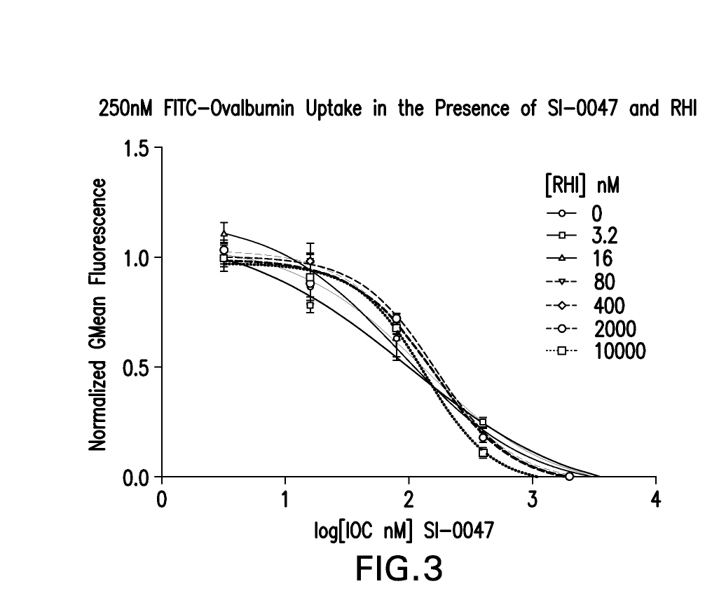

NR8383 were incubated, as described above, with a constant concentration (250 nM) of FITC-ovalbumin and various mixtures of SI-0047 and RHI at varying concentrations. The data in FIG. 3 show that the ability of SI-0047 to inhibit FITC-ovalbumin uptake was independent of the amount of RHI present. This indicates that the insulin receptor pathway does not play a role in the ability of the conjugate to be taken up by the mannose receptor pathway (i.e., there is no cooperativity between the two pathways).

### Example 4

In order to determine whether exposure to conjugates affects the ability of macrophages to carry out their normal functions (i.e., responding to inflammatory stimuli), NR8383 were exposed to SI-0052 and then stimulated to produce an inflammatory response. NR8383 were seeded in gelatin coated 24 well culture plate. Cells were then incubated with varying concentrations of SI-0052 in culture medium. After 24 hours, this solution was removed and the cells washed 1× with Hank's balanced saline solution (HBSS). Cells were then stimulated with 10 ng/mL of LPS from E. coli 0111:B4 (Sigma) in culture medium. After 24 hours, cell culture supernatant was collected and assayed for various inflammatory cytokines (IL-1β, IL-6, IL-10, TNFα) using colorometric ELISA kits (R&D).

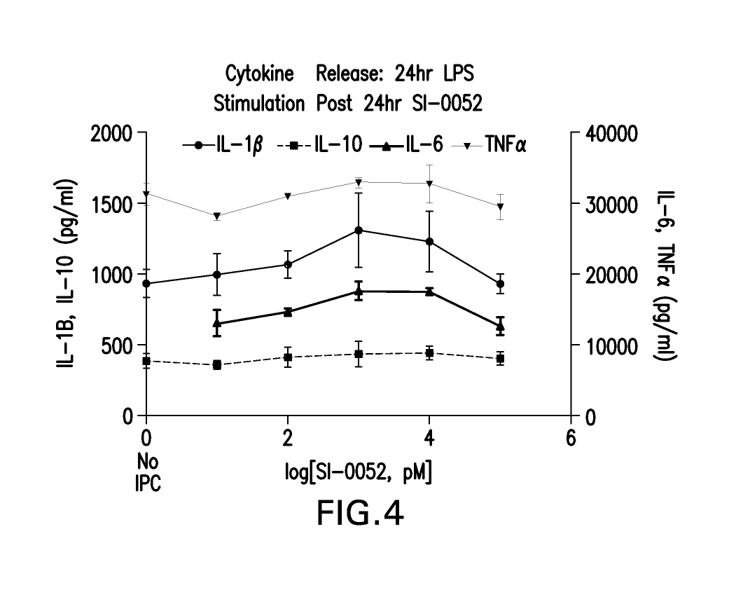

The data in FIG. 4 indicate that exposure to SI-0052, even at supra-physiological concentrations, did not prevent macrophages from producing an appropriate response to an inflammatory stimulus.

### REFERENCES

1. Loris, R. Principles of structures of animal and plant lectins. Biochimica et Biophysica Acta (BBA)-General Subjects 1572, 198-208 (2002).
2. Kilpatrick, D. C. Animal lectins: a historical introduction and overview. Biochimica et Biophysica Acta (BBA)-General Subjects 1572, 187-197 (2002).
3. East, L. & Isacke, C. M. The mannose receptor family. Biochimica et Biophysica Acta (BBA)-General Subjects 1572, 364-386 (2002).
4. Kerrigan, A. M. & Brown, G. D. C-type lectins and phagocytosis. Immunobiology 214, 562-575 (2009).
5. Apostolopoulos, V. & Ifc, M. Role of the mannose receptor in the immune response. Current molecular medicine 1, 469-474 (2001).
6. Taylor, M. E., Bezouska, K. & Drickamer, K. Contribution to ligand binding by multiple carbohydrate-recognition domains in the macrophage mannose receptor. Journal of Biological Chemistry 267, 1719 (1992).
7. Magnusson, S. & Berg, T. Extremely rapid endocytosis mediated by the mannose receptor of sinusoidal endothelial rat liver cells. Biochemical Journal 257, 651 (1989).

1. A method comprising:
exposing target cells expressing macrophage mannose receptor (MMR) to one or more candidate compounds;
determining whether the one or more candidate compounds are taken up into the target cells;
determining whether the uptake of the one or more candidate compounds is decreased in the presence of an inhibitor that binds MMR; and
selecting at least one candidate compound which exhibits decreased uptake in the presence of the inhibitor.

2. The method of claim 1, wherein the at least one selected candidate compound is a drug-saccharide conjugate.

3. The method of claim 1, wherein the at least one selected candidate compound is a saccharide, the method further comprising a step of conjugating the at least one selected candidate compound to a drug.

4. The method of claim 2 or 3, wherein the drug is an insulin molecule.

5. The method of claim 4, wherein the drug is wild-type human insulin.

6. The method of claim 2, wherein the drug-saccharide conjugate comprises at least two saccharides that are conjugated at different positions on the drug.

7. The method of claim 1, wherein the at least one selected candidate compound comprises at least one multivalent saccharide.

8. The method of claim 1, wherein the at least one selected candidate compound comprises a mannose residue.

9. The method of claim 1, wherein the at least one selected candidate compound comprises a glucose residue.

10. The method of claim 1, wherein the at least one selected candidate compound comprises a fucose residue.

11. The method of claim 1, wherein the target cells are macrophages.

12. The method of claim 1, wherein the inhibitor is mannan.

13. The method of claim 1, wherein the inhibitor is α-methyl mannose.

14. The method of claim 1, wherein the inhibitor is glucose.

15. The method of claim 1, wherein the one or more candidate compounds comprise a label.

16. The method of claim 15, wherein the label is fluorescent.

17. The method of claim 1, wherein the step of determining whether the uptake of the one or more candidate compounds is decreased in the presence of the inhibitor is performed at a plurality of candidate compound concentrations.

18. The method of claim 1, wherein the step of determining whether the uptake of the one or more candidate compounds is decreased in the presence of the inhibitor is performed at a plurality of inhibitor concentrations.

19. A method comprising:
exposing target cells expressing macrophage mannose receptor (MMR) to a control compound that binds MMR and is internalized into the target cells;
determining whether the presence of one or more candidate compounds decreases the uptake of the control compound; and
selecting at least one candidate compound which decreases the uptake of the control compound.

20. The method of claim 19, wherein the at least one selected candidate compound is a drug-saccharide conjugate.

21-38. (canceled)
